(19) United States
(12) Patent Application Publication
(10) Pub. No.: US 2021/0277347 A1
(43) Pub. Date: Sep. 9, 2021

(54) CELL CULTURE VESSEL AND CELL CHIP

(71) Applicants: Tomoaki NAKAYAMA, Tokyo (JP); Shinnosuke KOSHIZUKA, Kanagawa (JP); Takeru SUZUKI, Saitama (JP); Ryuya MASHIKO, Tokyo (JP); Minoru KO, Baltimore, MD (US)

(72) Inventors: Tomoaki NAKAYAMA, Tokyo (JP); Shinnosuke KOSHIZUKA, Kanagawa (JP); Takeru SUZUKI, Saitama (JP); Ryuya MASHIKO, Tokyo (JP); Minoru KO, Baltimore, MD (US)

(21) Appl. No.: 17/189,401

(22) Filed: Mar. 2, 2021

Related U.S. Application Data
(60) Provisional application No. 62/985,901, filed on Mar. 6, 2020.

(30) Foreign Application Priority Data
Dec. 28, 2020 (JP) 2020-218862

(51) Int. Cl. C12M 1/32; C12M 1/12; C12M 1/34

(52) U.S. Cl. CPC C12M 23/12; C12M 41/36; C12M 25/06

(57) ABSTRACT

A cell culture vessel includes: a base member having a plurality of recesses; and a frame member having through-holes and configured to be detachable from and attachable to the base member, wherein when the frame member is stacked on the base member, the plurality of recesses and the through-holes communicate with each other, the frame member includes a plurality of kinds of frame members, and the plurality of kinds of frame members have mutually different combinations of the through-holes communicating with the plurality of recesses.

### TECHNICAL FIELD

The present invention relates to a cell culture vessel and a cell chip.

### BACKGROUND ART

In recent years, there has been an increasing demand for a tool for evaluating toxicity or drug efficacy using live cells in vitro. As one of the reasons therefor, background knowledge indicates that there is a need for a testing method that substitutes for animal experiments by promoting the 3Rs (“Replacement”, “Reduction”, and “Refinement”) of animal experiments. In regard to this problem, experiments that use live cells in vitro have many advantages, such as reduction of cost for laboratory animals and shortening of the test time.

Furthermore, there is a technique of dispensing cells, collected from a plurality of kinds of patients as the cells to be used for the above-described tool, into a single culture vessel, and performing comparison and evaluation of a test, and in order to make these evaluations more efficient, a vessel in which the recesses formed on the culture vessel are made small and integrated has been developed. In this integrated vessel, since the volume of medium that can be accommodated in the recesses is small, the medium may be affected by drying, and cell culture may become unstable. As a culture vessel that solves this problem, a culture vessel which uses inner walls that are erected around a group of recesses and serve as secondary recesses that can hold a solution has already been developed (see, for example, Japanese Patent No. 4576539 and the like).

Generally, in a toxicity or drug efficacy evaluation test using live cells in vitro, solutions having different compositions, such as a medium and a drug solution, are used in various steps such as a cell culturing step and an evaluation step of adding a drug to cells. When a series of steps from the cell culture to evaluation are carried out in the same culture vessel, the combination of recesses that share a medium in the culturing step may differ from the combination of recesses that share a drug solution in the evaluation step. However, in the culture vessel described in Japanese Patent No. 4576539 and the like, since the combination of recesses that share a solution is fixed, the combination of the recesses that share a solution cannot be freely changed according to the purpose.

### SUMMARY OF THE INVENTION

It is an object of the present invention to provide a cell culture vessel in which the combination of recesses sharing a solution can be freely changed.

According to an aspect of the present invention, a cell culture vessel is provided, including a base member having a plurality of recesses; a frame member having through-holes and configured to be detachable from and attachable to the base member. When the frame member is stacked on the base member, the plurality of recesses and the through-holes communicate with each other, the frame member includes a plurality of kinds of frame members, and the plurality of kinds of frame members have mutually different combinations of the through-holes communicating with the plurality of recesses.

According to the cell culture vessel of the present invention, a cell culture vessel in which the combination of recesses sharing a solution can be freely changed can be provided.

### BRIEF DESCRIPTION OF THE DRAWINGS

FIG. 1 is an exploded perspective view of a cell culture vessel according to a first embodiment of the present invention.

FIG. 2 is a cross-sectional view of the cell culture vessel cut along a plane that passes through the line II-II shown in FIG. 1.

FIG. 3 is a perspective view of the cell culture vessel according to the first embodiment of the present invention.

FIG. 4 is a cross-sectional view of the cell culture vessel cut along a plane that passes through the line IV-IV shown in FIG. 3.

FIG. 5 is an exploded perspective view of a cell culture vessel according to a second embodiment of the present invention.

FIG. 6 is a plan view of the cell culture vessel according to the second embodiment of the present invention.

FIG. 7 is a cross-sectional view of the cell culture vessel cut along a plane that passes through the line VII-VII shown in FIG. 6.

FIG. 8 is an exploded perspective view of a cell culture vessel according to a third embodiment of the present invention.

FIG. 9 is a plan view of the cell culture vessel according to the third embodiment of the present invention.

FIG. 10 is a cross-sectional view of the cell culture vessel that has been cut along a plane passing through the line X-X shown in FIG. 9.

FIG. 11 is an exploded perspective view of a cell culture vessel according to a fourth embodiment of the present invention.

FIG. 12 is a plan view of the cell culture vessel according to the fourth embodiment of the present invention.

FIG. 13 is a cross-sectional view of the cell culture vessel cut along a plane that passes through the line XIII-XIII shown in FIG. 12.

FIG. 14 is an exploded perspective view of a cell culture vessel according to a fifth embodiment of the present invention.

FIG. 15 is a cross-sectional view of the cell culture vessel according to the fifth embodiment of the present invention.

FIG. 16 is an exploded perspective view of a cell culture vessel according to a sixth embodiment of the present invention.

FIG. 17 is a cross-sectional view of the cell culture vessel cut along a plane that passes through the line XVII-XVII shown in FIG. 16.

FIG. 18 is a diagram showing a method for evaluating a drug as a usage example for the cell culture vessel according to an embodiment of the present invention.

FIG. 19 is a diagram showing a method for evaluating a drug as a usage example for the cell culture vessel according to an embodiment of the present invention.

FIG. 20 is an exploded perspective view of a cell chip according to an embodiment of the present invention.

FIG. 21 is a plan view of the cell chip according to an embodiment of the present invention.

FIG. 22 is a cross-sectional view of the cell chip cut along a plane that passes through the line XXII-XXII shown in FIG. 21.

### EMBODIMENTS FOR CARRYING OUT THE INVENTION

Hereinafter, the cell culture vessel and cell chip according to the embodiments of the present invention will be described with reference to the drawings.

The embodiments that will be described below are merely examples for facilitating the understanding of the present invention and are not intended to limit the present invention. That is, the shapes, dimensions, dispositions, and the like of the members that will be described below can be changed and improved while maintaining the gist of the present invention, and the present invention includes equivalents thereof.

Furthermore, in all of the drawings, similar constituent elements will be assigned with similar reference numerals, and any overlapping description will be omitted as appropriate.

### <Cell Culture Vessel>

### First Embodiment

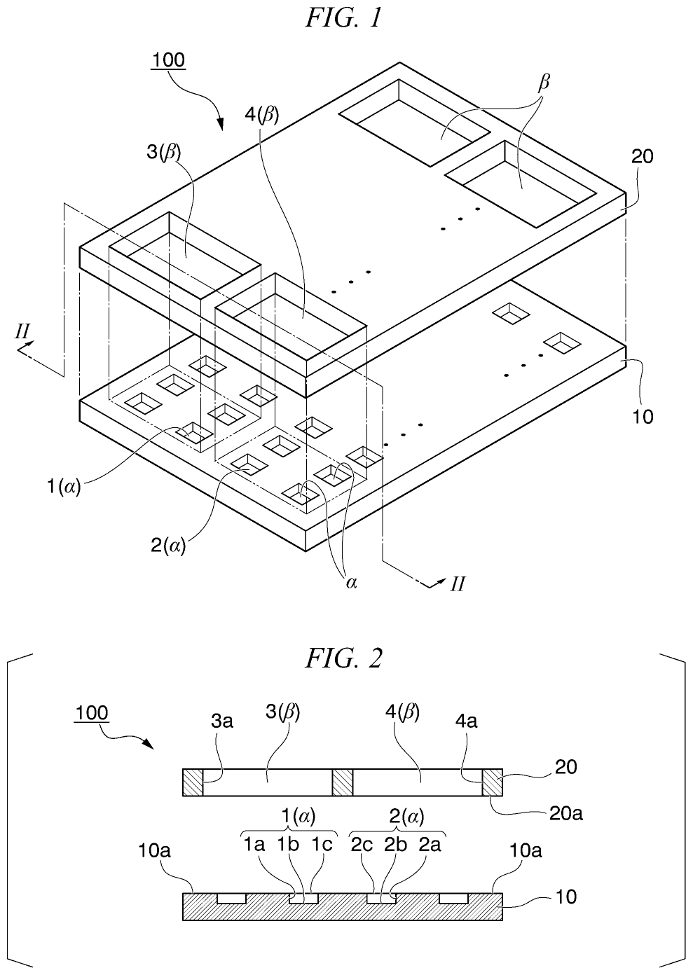

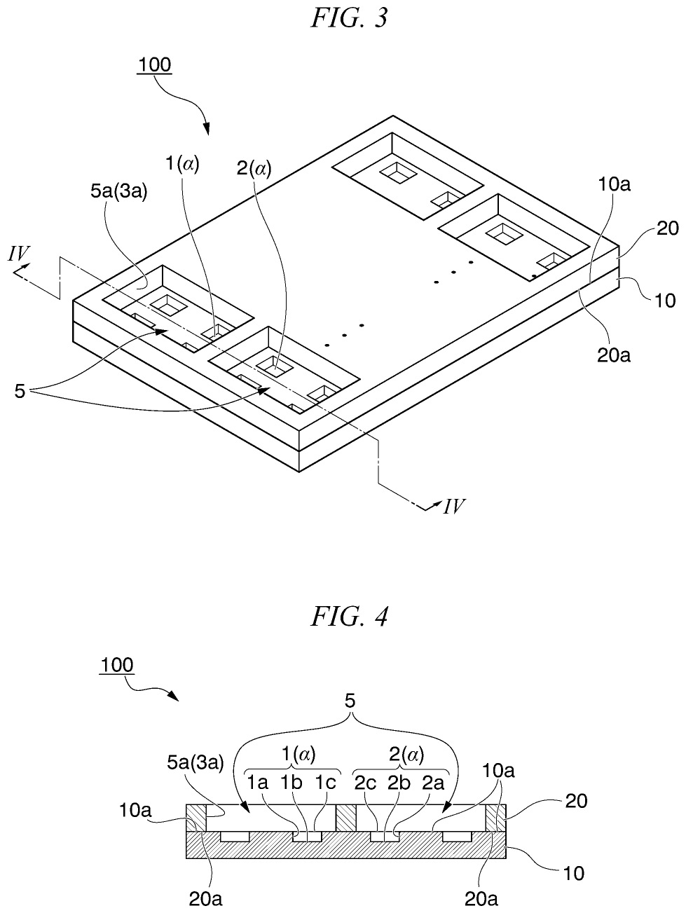

FIGS. 1 to 4 are diagrams illustrating a cell culture vessel 100 according to a first embodiment of the present invention. FIG. 1 is an exploded perspective view of the cell culture vessel 100. FIG. 2 is a cross-sectional view of the cell culture vessel 100 cut along a plane that passes through the line II-II shown in FIG. 1. FIG. 3 is a perspective view of the cell culture vessel 100. FIG. 4 is a cross-sectional view of the cell culture vessel 100 cut along a plane that passes through the line IV-IV of FIG. 3.

As shown in FIG. 1, the cell culture vessel 100 includes a base member 10; and a frame member 20 that is used in a state of being stacked on a top surface 10a of the base member 10.

### (Recesses and Through-Holes)

As shown in FIG. 1 and FIG. 2, the base member 10 has a plurality of recesses α including a first recess 1 and a second recess 2 on the top surface 10a of the base member. The frame member 20 has a plurality of through-holes β including a first through-hole 3 and a second through-hole 4. The first through-hole 3 communicates with two or more recesses α including the first recess 1, and the second through-hole 4 communicates with two or more recesses α including the second recess 2.

Hereinafter, a surface of the frame member 20, the surface being disposed to face the top surface 10a of the base member 10, is referred to as a bottom surface 20a (or a joining side principal surface 20a) of the frame member 20.

As shown in FIG. 3 and FIG. 4, the cell culture vessel 100 can be used by having the bottom surface (joining side principal surface) 20a of the frame member 20 on the top surface 10a of the base member 10. The frame member 20 is configured to be detachable from and attachable to the base member 10. By using the cell culture vessel while having the frame member 20 on the base member 10, cells to which solutions having different compositions have been added can be cultured simultaneously.

Furthermore, as shown in FIG. 4, the through-holes β of the frame member 20 form recess clusters 5, which are each surrounded by two or more recesses α and a through-hole β. Thereby, a solution in an amount exceeding the volume of the recesses can be held, drying can be prevented, and the control and management of the liquid level can be easily achieved.

As shown in FIG. 2 and FIG. 4, the first recess 1 and the second recess 2 have an opening 1c and an opening 2c, respectively, on the top surface 10a of the base member 10 and also have a bottom surface 1b and a bottom surface 2b, as well as an inner wall 1a and an inner wall 2a having a predetermined depth and configured to surround the bottom surface 1b and the bottom surface 2b. Other recesses are also configured similarly to the first recess 1 and the second recess 2.

All the recesses α included in the base member 10 are formed so as to extend from the top surface 10a of the base member 10 such that the axes of the recesses α formed in a tubular shape are parallel to one another.

All the through-holes β included in the frame member 20 are formed so as to penetrate through the thickness of the frame member 20 such that the axes of the through-holes β formed in a tubular shape are parallel to one another.

The frame member 20 is stacked on the base member 10 such that each through-hole 13 is disposed coaxially with a recess α of the base member 10. By superposing the frame member 20 on the base member 10, the through-hole β can communicate with the recess α.

With regard to the recess α and the through-hole β that communicate with each other when the frame member 20 is superposed on the base member 10, the recess and the through-hole are configured to have shapes and sizes that allow the recess α that communicates with the interior of the through-hole β to be completely exposed as viewed in plan view.

Hereinafter, a recess α formed in the base member 10, or a recess cluster 5 surrounded by two or more recesses α and a through-hole β, may be simply referred to as a “well”.

Any number of recesses α may be formed in the base member 10, and for example, the number may be 4, 6, 12, 24, 48, 96, 384, or 1536.

The shape of the recess α and the through-hole β as viewed in plan view is not particularly limited and can be appropriately selected according to the purpose, and examples include a circular shape, an approximately circular shape, a triangular shape, and a quadrangular shape.

For example, in a case where the recess α has a circular shape as viewed in plan view, the inner diameter of the recess α is preferably 750 μm or more and 6,000 μm or less, and more preferably 1,500 μm or more and 3,000 μm or less.

The disposition of a plurality of recesses α and a plurality of through-holes β is not particularly limited and can be appropriately selected according to the purpose, and for example, the disposition may be arranged to correspond to a pattern shape capable of forming a desired histological body.

The bottom surface of the recess α is not particularly limited and can be appropriately selected according to the purpose, and examples include a flat bottom surface and a bottom surface configured to have a curved surface such that a cross section thereof is convex downward.

It is preferable that the volume of the recess α be 10 μL or less, and the volume of the recess cluster 5 be 10 μL or more and 100 μL or less. Furthermore, it is more preferable that the volume of the recess α be 100 nL or more and 10 μL or less, and the volume of the recess cluster 5 be 10 μL or more and 100 μL or less.

Even for a minute recess in which the volume of the recess α is less than or equal to the above-described upper limit, when the volume of the recess cluster 5 is more than or equal to the above-described lower limit, the volume of a solution that can be contained can be made larger than the volume of the solution in the case of having recesses only, and the solution is not likely to be affected by drying. In addition, control and management of the liquid level can be easily achieved, and cells can be stably cultured.

Furthermore, it is preferable that each of the through-holes included in the frame member 20 communicates with three or more recesses. Thereby, various evaluations can be performed using at least three samples of cells cultured under the same conditions.

In a case where cells are seeded by an inkjet method, the inner bottom surface of the recess α serves as a landing surface for the droplets of a cell suspension.

### (Base Member and Frame Member)

The base member 10 and the frame member 20 are each plate-shaped members.

The base member 10 and the frame member 20 may be formed of any material as long as the material is not toxic to cells; however, an elastic material, glass, a ceramic, a metal material such as stainless steel, and the like are preferable. Examples of the elastic material include synthetic resins such as a cycloolefin, polystyrene, polyethylene, polypropylene, polycarbonate, polyimide, polyacetal, polyester (polyethylene terephthalate or the like), polyurethane, polysulfone, polyacrylate, polymethacrylate (polymethyl methacrylate (PMMA) or the like), and polyvinyl; silicon-based resins such as PDMS (Poly-Dimethylsiloxane); synthetic rubbers such as EPDM (Ethylene Propylene Diene Monomer); and natural rubber.

Regarding the base member 10 and the frame member 20, these materials can be used singly or in combination of two or more kinds thereof.

Regarding the material constituting the base member 10, particularly the material forming the recess α, among the above-mentioned materials, a translucent material is preferred. When the material constituting the recess α is a translucent material, the cells cultured in the base member 10 can be easily observed by optical means such as a microscope.

The height of the base member 10 can be adjusted to, for example, more than 0 nm and 12,000 μm or less and can be adjusted to 100 μm or more and 5,000 μm or less.

The height of the base member as used herein means the height of the entire base member, and for example, the height of a base member composed of a plurality of layers means the total height of all the layers constituting the base member.

The height of the frame member can be adjusted to, for example, 100 μm or more and 12,000 μm or less and can be adjusted to 1,000 μm or more and 12,000 μm or less.

The height of the frame member as used herein means the height of the entire frame member, and for example, the height of a frame member composed of a plurality of layers means the total height of all the layers constituting the frame member.

Upon forming the base member 10 and the frame member 20, any processing method that is selected according to the purpose can be used. That is, for example, the base member and the frame member can be formed by perforation processing using a machining center or the like, optical microfabrication processing using a laser or the like, processing by photolithography, etching processing, or embossing processing. Furthermore, the base member and the frame member can be formed by, for example, injection molding, press molding, or stereolithography.

Among these, laser processing is preferred as a method for forming the recess α and the through-hole β. When the method for forming the recess α and the through-hole β is laser processing, the pattern shape of wells can be easily and finely formed. In addition, since processing is achieved without direct contact with the base member 10 and the frame member 20, it is advantageous from the viewpoint that biocompatibility is easily obtained.

It is preferable that the base member 10 and the frame member 20 be sterilized because cells come into contact with the members.

The cell culture vessel of the present embodiment includes, in addition to the frame member 20 shown in the cell culture vessel according to the first embodiment, a plurality of kinds of frame members such as a second frame member 30 and an outer frame member 40, as shown in the cell culture vessels according to the second to fourth embodiments that will be described later. A plurality of kinds of these frame members differ from each other in the combination of through-holes μ that communicate with a plurality of recesses α. Therefore, the combination of recesses α sharing a solution can be freely changed by changing the frame member to be superposed on the base member 10.

### Second Embodiment

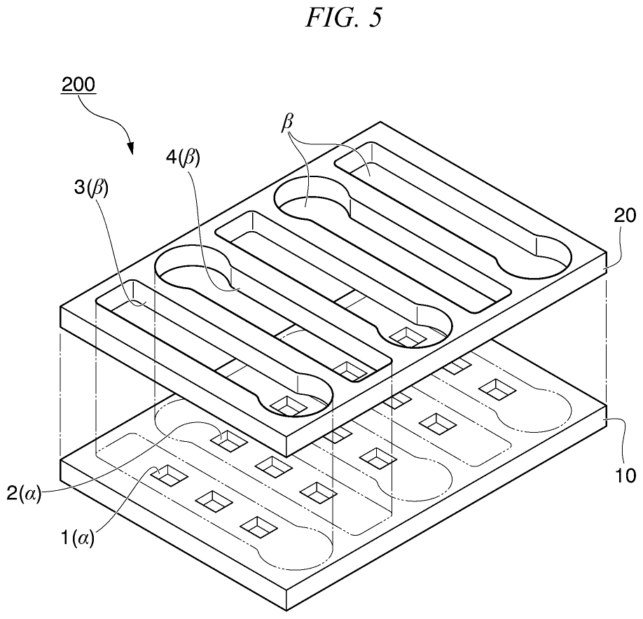

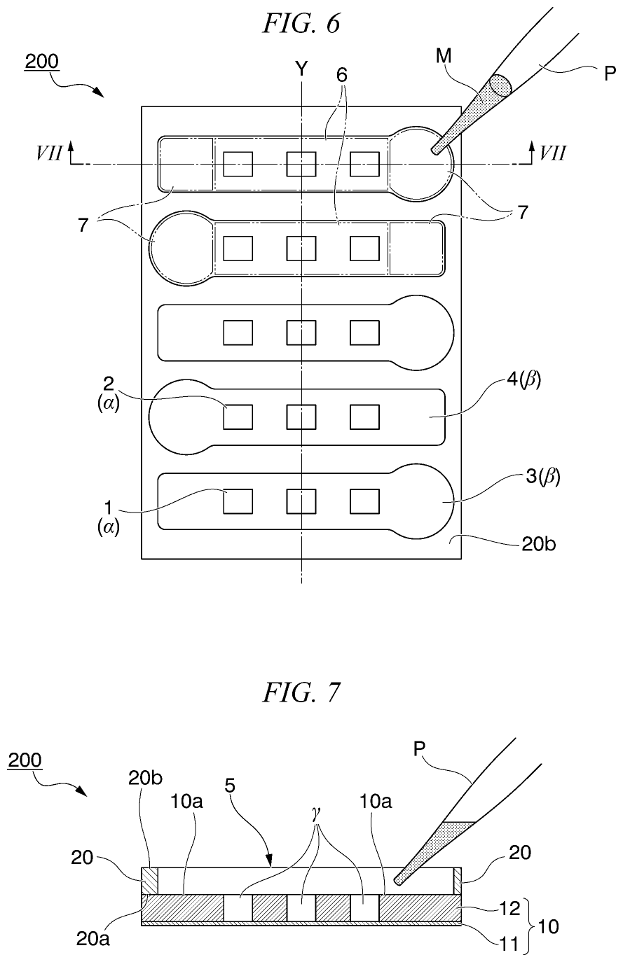

FIGS. 5 to 7 are diagrams illustrating a cell culture vessel 200 according to a second embodiment of the present invention. FIG. 5 is an exploded perspective view of the cell culture vessel 200. FIG. 6 is a plan view of the cell culture vessel 200. FIG. 7 is a cross-sectional view of the cell culture vessel 200 cut along a plane that passes through the line VII-VII of FIG. 6. The cell culture vessel 200 shown in FIGS. 5 to 7 is different from the cell culture vessel 100 shown in FIGS. 1 to 4 from the viewpoint that the shape of a through-hole β of the frame member 20 as viewed in plan view is a shape combining an approximately circular shape and a rectangular shape.

As shown in FIG. 6, a plurality of recesses α and a portion of a top surface 10a of a base member 10 are exposed inside a through-hole β as viewed in plan view, and the through-hole has a first exposed part 6 in which a plurality of recesses α are exposed; and a second exposed part 7 which is formed contiguously to the first exposed part 6 and in which only the top surface 10a of the base member 10 is exposed. It is preferable that the area of the top surface 10a of the base member 10 to be exposed at the second exposed part 7 be larger than the area of the recess as viewed in plan view. By adopting the above-described configuration, when a solution is added using a pipette P, the influence of the liquid flow on the cells in the recess α can be reduced, and detachment of the cells can be prevented.

Specifically, the area of the top surface 10a of the base member 10 to be exposed at the second exposed part 7 can be adjusted to, for example, 1.1 or more times, 1.2 or more times, or 1.5 or more times, the area of the recess α as viewed in plan view.

Furthermore, as shown in FIG. 6, in the frame member 20, a plurality of through-holes β are arranged along an arrangement axis Y extending in one direction. With regard to two adjoining through-holes β along the arrangement axis Y, for example, a first through-hole 3 and a second through-hole 4, it is preferable that the second exposed part 7 of the first through-hole 3 and the second exposed part 7 of the second through-hole be disposed to have the arrangement axis Y interposed therebetween. By adopting the above-described configuration, more through-holes can be formed in the top surface 20b of the frame member 20.

Furthermore, the top surface 10a of the base member 10 to be exposed at the second exposed part 7 may have a pit. By adopting the above-described configuration, the influence of a liquid flow can be further reduced upon pipetting a solution.

Furthermore, as shown in FIG. 7, the base member 10 may be composed of a substrate 11 and a recess-forming frame member 12. The recess-forming frame member 12 is used in a state of being stacked on the substrate 11, has a plurality of through-holes γ for recesses corresponding to a plurality of recesses α, and the space surrounded by the substrate 11 and the through-holes γ for recesses forms the recess α. Since the base member 10 is composed of the substrate 11 and the recess-forming frame member 12, the recess-forming frame member 12 can be separated from the substrate 11, and then the cells disposed on the top surface of the substrate 11 can be directly subjected to staining to be directly observed and evaluated using a microscope or the like.

### Third Embodiment

The cell culture vessel of the present embodiment includes a plurality of kinds of frame members, and for example, in a case where the cell culture vessel includes the first frame member and the second frame member, the first frame member and the second frame member have mutually different combinations of two or more recesses α that are exposed inside a through-hole β as viewed in plan view. Thus, the combination of recesses that share a solution can be freely changed by changing the frame member to be superposed on the base member according to the use application. As a specific use application, the first frame member can be used for cell culture, while the second frame member can be used for drug evaluation. Alternatively, the first frame member can be used for drug evaluation, while the second frame member can be used for cell culture.

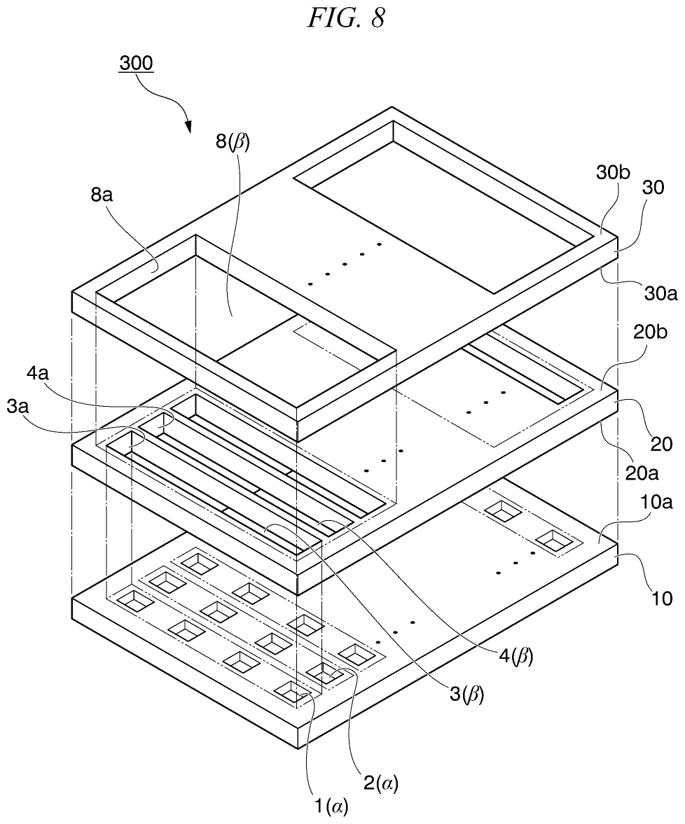

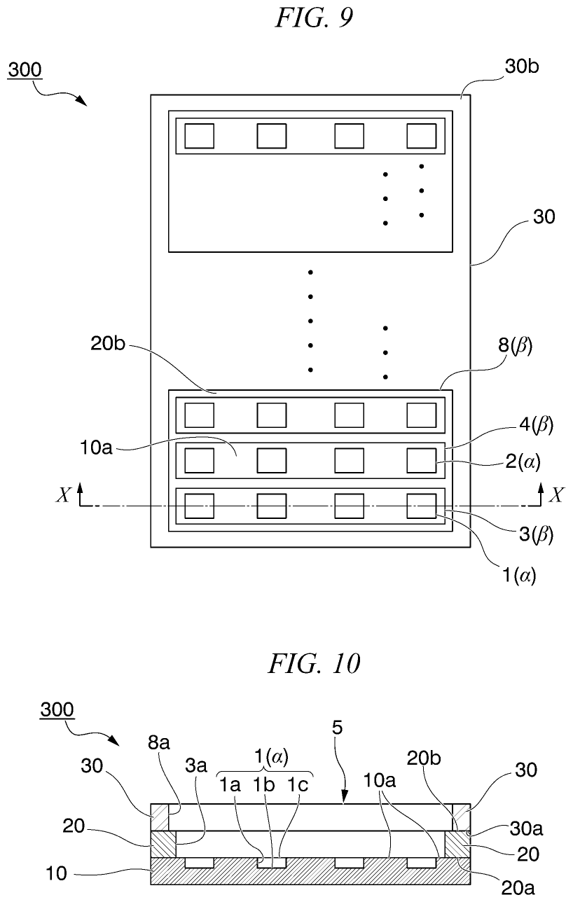

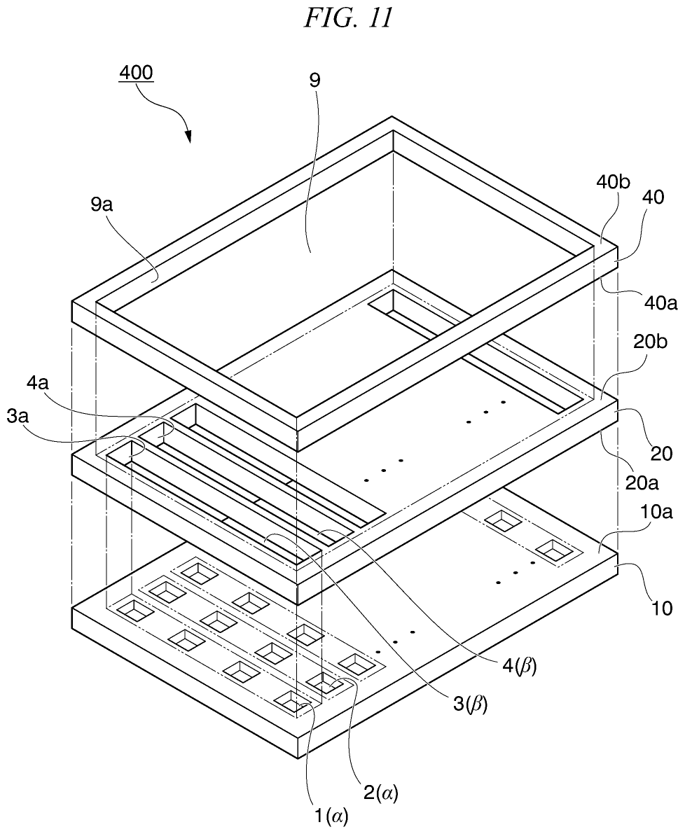

FIGS. 8 to 10 are diagrams illustrating a cell culture vessel 300 according to a third embodiment of the present embodiments. FIG. 8 is an exploded perspective view of the cell culture vessel 300. FIG. 9 is a plan view of the cell culture vessel 300. FIG. 10 is a cross-sectional view of the cell culture vessel 300 cut along a plane that passes through the line X-X of FIG. 9. The cell culture vessel 300 shown in FIGS. 8 to 10 is different from the cell culture vessel 100 shown in FIGS. 1 to 4 from the viewpoint that the cell culture vessel 300 includes two frame members.

As shown in FIG. 8, a first frame member 20 is used in a state of being stacked on a top surface 10a of the base member 10, and a second frame member 30 is used in a state of being stacked on a top surface 20b of the first frame member 20. As shown in FIG. 9, it is preferable that when the first frame member 20 and the second frame member 30 are stacked on the base member 10, inside a third through-hole 8 of the second frame member 30, two or more through-holes β including a first through-hole 3 and a second through-hole 4 among a plurality of through-holes β included in the first frame member 20 be completely exposed as viewed in plan view. In the cell culture vessel 300 shown in FIGS. 8 to 10, the combination of recesses sharing a solution can be freely changed by using the frame members in a state of being separated apart in order from the top. Furthermore, for example, when the base member 10, the first frame member 20, and the second frame member 30 are stacked, cell culture is performed, and when the base member and the first frame member 20 are stacked while the second frame member 30 is separated out, drug evaluation can be performed.

Each of the first through-hole 3 and the second through-hole 4 included in the first frame member 20 may be respectively disposed such that a plurality of recesses α including a first recess 1 or a second recess 2 arranged vertically and horizontally as shown in FIGS. 1 to 4 as viewed in plan view are completely exposed. The through-holes may also be disposed such that as shown in FIG. 9, a plurality of recesses α arranged in a horizontal row are completely exposed, or a plurality of recesses α arranged in a vertical row are completely exposed.

As shown in FIG. 9, a third through-hole 8 included in the second frame member 30 may be disposed such that two or more through-holes β including the first through-hole 3 and the second through-hole 4 arranged in a vertical row as viewed in plan view are completely exposed, or the third through-hole may be disposed such that two or more through-holes β arranged in a horizontal row in the first frame member 20 are completely exposed.

As shown in FIG. 10, when the first frame member 20 and the second frame member 30 are stacked on the base member 10, the through-holes β of the second frame member 30 form a recess cluster 5 that is surrounded by two or more recesses α, a through-hole β included in the first frame member 20, and a through-hole β included in the second frame member 30.

### Fourth Embodiment

The cell culture vessel of the present embodiment may further include an outer frame member.

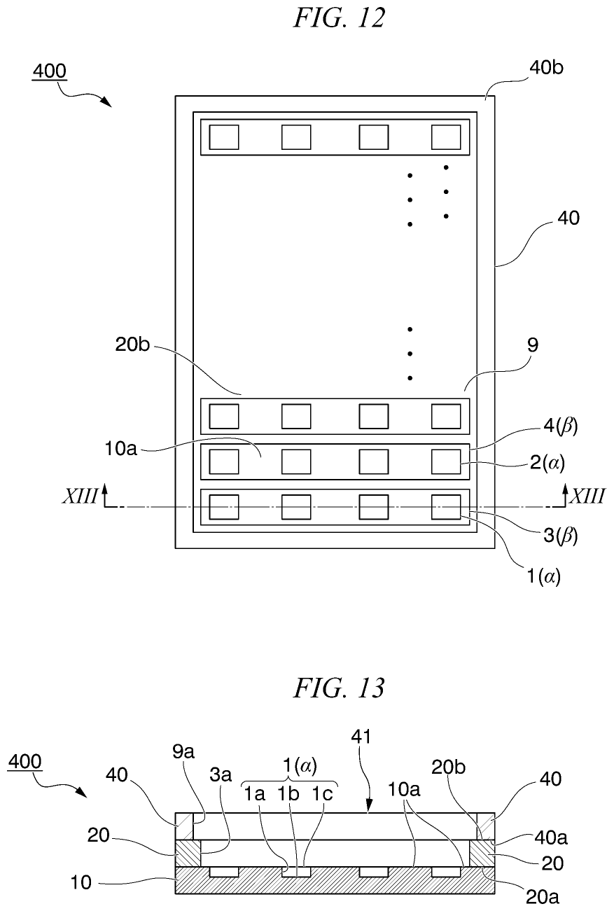

FIGS. 11 to 13 are diagrams illustrating a cell culture vessel 400 according to a fourth embodiment of the present embodiments. FIG. 11 is an exploded perspective view of the cell culture vessel 400. FIG. 12 is a plan view of the cell culture vessel 400. FIG. 13 is a cross-sectional view of the cell culture vessel 400 cut along a plane that passes through the line XIII-XIII of FIG. 12. The cell culture vessel 400 shown in FIGS. 11 to 13 is different from the cell culture vessel 100 shown in FIGS. 1 to 4 from the viewpoint that the cell culture vessel 400 further includes the outer frame member 40. By including the outer frame member 40, the cell culture vessel of the present embodiment can hold a larger amount of a solution in the cell culture vessel and can prevent drying. In addition, all cells in the cell culture vessel can be cultured in a common medium.

As shown in FIG. 11, the outer frame member 40 is configured to be detachable from and attachable to the frame member 20 and is used in a state of being stacked on the top surface 20b of the frame member 20. Furthermore, the outer frame member 40 is configured to be detachable from and attachable to the base member 10 and can be used in a state of being stacked on the top surface 10a of the base member 10. The outer frame member 40 has one communicating hole 9.

As shown in FIG. 12, when the frame member 20 and the outer frame member 40 are stacked on the base member 10, inside the communicating hole 9 included in the outer frame member 40, a plurality of recesses α included in the base member 10 and a plurality of through-holes β included in the frame member 20 are all completely exposed as viewed in plan view.

As shown in FIG. 13, when the frame member 20 and the outer frame member 40 are stacked on the base member 10, the communicating hole 9 communicates with a plurality of recesses α and a plurality of through-holes β, and thereby a liquid reservoir 41 surrounded by the plurality of recesses α, a plurality of through-holes β, and the communicating hole 9 is formed.

### (Outer Frame Member)

The outer frame member 40 is a plate-shaped member.

Regarding the material constituting the outer frame member 40, materials similar to those exemplified as the materials constituting the base member 10 and the frame member 20 may be mentioned. Furthermore, as a method for forming the outer frame member 40, a method similar to the method exemplified as the method for forming the base member 10 and the frame member 20 may be mentioned.

The height of the outer frame member can be adjusted to, for example, 100 μm or more and 12,000 μm or less and can be adjusted to 1,000 μm or more and 5,000 μm or less.

Incidentally, the height of the outer frame member as used herein means the height of the entire outer frame member, and for example, the height of an outer frame member composed of a plurality of layers means the total height of all the layers constituting the outer frame member.

### Fifth Embodiment

A cell culture vessel of the present embodiment may further include a lid member on the uppermost surface.

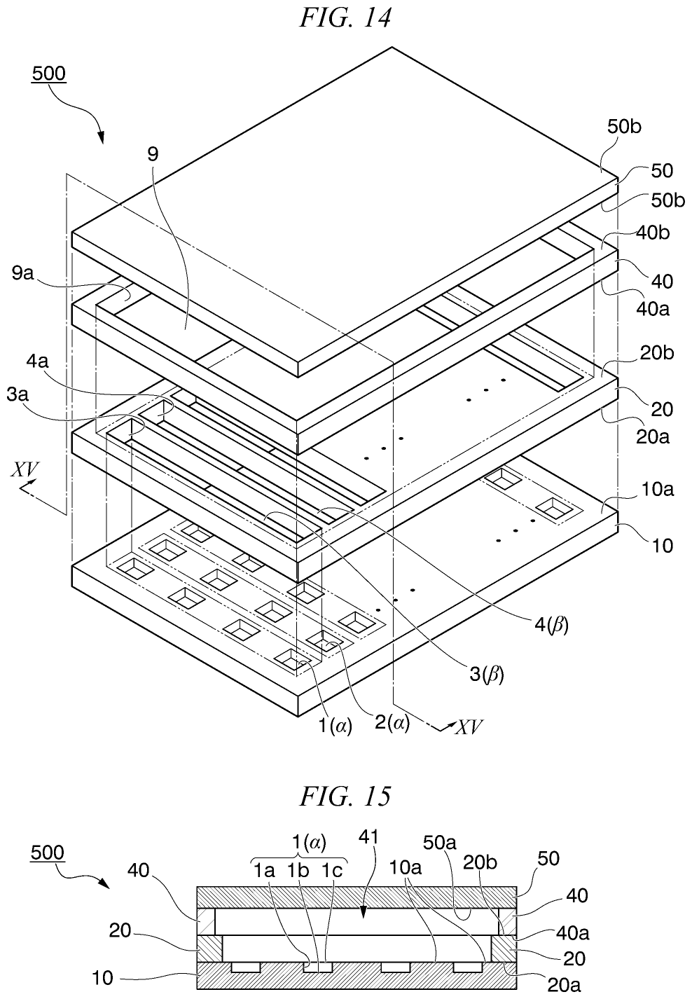

FIGS. 14 and 15 are diagrams illustrating a cell culture vessel 500 according to a fifth embodiment of the present invention. FIG. 14 is an exploded perspective view of the cell culture vessel 500. FIG. 15 is a cross-sectional view of the cell culture vessel 500. The cell culture vessel 500 shown in FIGS. 14 and 15 is different from the cell culture vessel 400 shown in FIGS. 11 to 13 from the viewpoint that the cell culture vessel 500 further includes a lid member 50. By including the lid member 50, the cell culture vessel of the present embodiment can prevent drying and can prevent liquid leakage during transfer of the vessel.

The lid member 50 is configured to be detachable from and attachable to the frame member 20 and is used in a state of being stacked on the top surface 20b of the frame member 20. Furthermore, the lid member 50 may also be configured to be detachable from and attachable to the outer frame member 40 and used in a state of being stacked on a top surface 40b of the outer frame member 40.

As shown in FIG. 15, by superposing the lid member 50 on the top surface 40b of the outer frame member 40, the liquid reservoir 41 is liquid-tightly sealed by the lid member 50.

### (Lid Member)

The lid member 50 is a plate-shaped member.

Regarding the material constituting the lid member 50, materials similar to those exemplified as the materials constituting the base member 10 and the frame member 20 may be mentioned. Furthermore, as a method for forming the lid member 50, a method similar to the method exemplified as the method for forming the base member 10 and the frame member 20 may be mentioned.

The height of the lid member can be adjusted to, for example, 100 μm or more and 12,000 μm or less and can be adjusted to 1,000 μm or more and 5,000 pin or less.

The height of the lid member as used herein means the height of the entire lid member, and for example, the height of the lid member composed of a plurality of layers means the total height of all the layers constituting the lid member.

### Sixth Embodiment

In the cell culture vessel of the present embodiment, the base member 10 and the frame member 20 can each include a portion forming a joining part at a corresponding position. By including the portion forming the joining part, the base member 10 and the frame member 20 can be easily positioned and superposed. Furthermore, when the cell culture vessel is transferred, the base member 10 and the frame member 20 can be stably carried without being displaced.

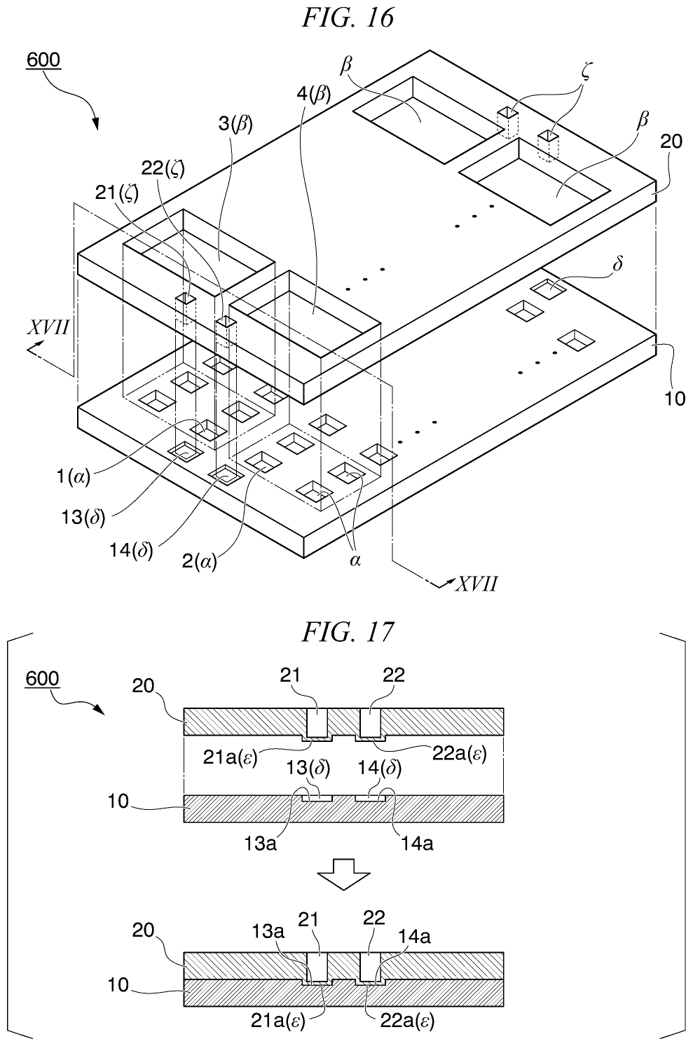

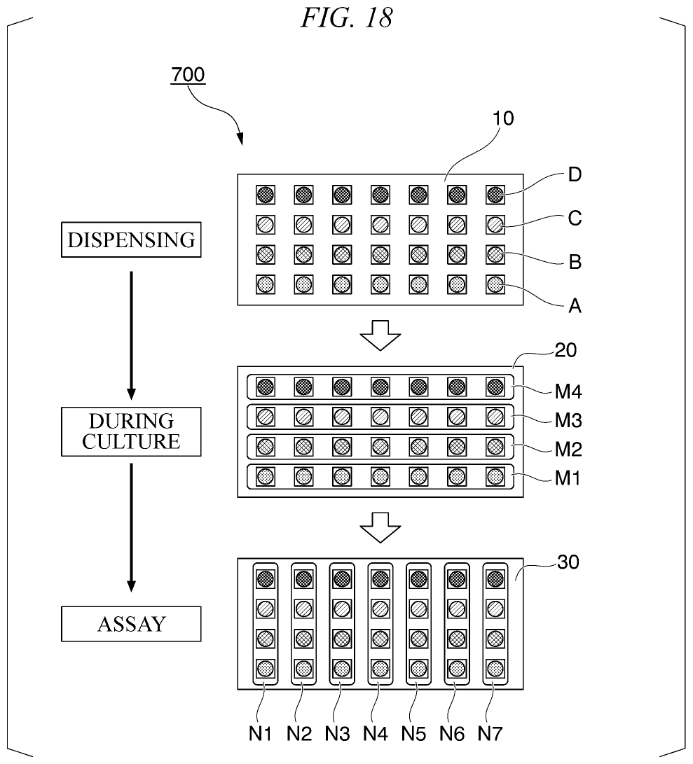

FIGS. 16 and 17 are diagrams illustrating a cell culture vessel 600 according to a sixth embodiment of the present invention. FIG. 16 is an exploded perspective view of the cell culture vessel 600 according to the sixth embodiment of the present invention. FIG. 17 is a cross-sectional view of the cell culture vessel 600 cut along a plane that passes through the line XVII-XVII shown in FIG. 16. The cell culture vessel 600 shown in FIGS. 16 and 17 is different from the cell culture vessel 100 shown in FIGS. 1 to 4 from the viewpoint that the cell culture vessel 600 further includes joining parts. A joining part in FIGS. 16 to 17 represents a fitting structure formed by a joining recess δ and a joining protrusion ε, which are disposed at positions where they can be fitted to each other.

As shown in FIGS. 16 to 17, the base member 10 has two or more joining recesses δ including a first joining recess 13 and a second joining recess 14 on the top surface 10a of the base member. The frame member 20 has two or more joining protrusions ε including a first joining protrusion 21a and a second joining protrusion 22a at positions corresponding to the joining recesses on the bottom surface (joining side principal surface) 20a. When the base member 10 and the frame member 20 are stacked, the first joining recess 13 and the first joining protrusion 21a are fitted together, and the second joining recess 14 and the second joining protrusion 22a are fitted together, thus each forming joining parts.

With regard to the cell culture vessel 600 shown in FIGS. 16 and 17, the case in which the base member 10 has a joining recess δ and the frame member 20 has a joining protrusions has been described as an example; however, the embodiment is not limited to this, and it is also acceptable that the base member 10 have a joining protrusion ε, while the frame member 20 have a joining recess δ. Furthermore, the shape of the portion constituting the joining part is not limited to the recess-protrusion shape, as long as the shape is capable of joining when the base member 10 and the frame member 20 are stacked.

The positions of the joining recess δ and the joining protrusion ε on the top surface 10a of the base member 10 and the bottom surface 20a of the frame member 20 are not particularly limited; however, it is preferable that one or more joining recesses δ and joining protrusions ε be disposed at the four corners of the base member 10 and the frame member 20. Since one or more joining recesses δ and joining protrusions ε are disposed at the four corners, the base member 10 and the frame member 20 can be superposed and held in a more stable state.

As shown in FIGS. 16 and 17, it is preferable that the frame member 20 have two or more recesses ζ for solution trial, including a first recess 21 for trial and a second recess 22 for trial. These recesses ζ for solution trial can be used to trial-hit droplets of a cell suspension when cells are seeded by an inkjet method.

In the frame member 20, it is preferable that the first recess 21 for trial and the first joining protrusion 21a be disposed at overlapping positions as viewed in plan view, and the second recess 22 for trial and the second joining protrusion 22a be disposed at overlapping positions. By having the recesses for trial and the joining protrusions ε arranged at such positions, the top surface 20b and the bottom surface 20a of the frame member 20 having a limited size can be effectively utilized.

The embodiments described above may be carried out singly or in combination of two or more of these embodiments.

The cell culture vessel of the present embodiments is not limited to those shown in FIGS. 1 to 17, and a portion of the configurations shown in FIGS. 1 to 17 may be modified or deleted, or other configurations may be further added to those that have been hitherto described, to the extent that the effects of the present invention are not impaired.

For example, with regard to the cell culture vessel 300 shown in FIGS. 8 to 10, the first frame member 20 and the second frame member 30 can each include portions that form a joining part (for example, a joining recess δ and a joining protrusion ε) at corresponding positions.

For example, with regard to the cell culture vessel 400 shown in FIGS. 11 to 13, the frame member 20 and the outer frame member 40 can each include portions that form a joining part (for example, a joining recess δ and a joining protrusion ε) at corresponding positions.

For example, with regard to the cell culture vessel 500 shown in FIGS. 14 and 15, the frame member 20 and the outer frame member 40 can each include portions that form a joining part (for example, a joining recess δ and a joining protrusion ε) at corresponding positions, and the outer frame member 40 and the lid member 50 can each include portions that form a joining part (for example, a joining recess δ and a joining protrusion ε) at corresponding positions.

As these members include portions that form a joining part, the members can be easily positioned and superposed with each other. In addition, when the cell culture vessel is transported, these members can be stably transported without shifting.

### <Usage Examples for Cell Culture Vessel>

FIG. 18 is a diagram illustrating a method for evaluating a drug as a usage example for a cell culture vessel according to an embodiment of the present invention. In the usage example shown in FIG. 18, seeding, culture, and assaying of cells can be carried out by replacing the frame member used in the cell culture vessel according to the purpose.

Specifically, first, four kinds of cells, namely, first cells A, second cells B, third cells C, and fourth cells D, are seeded on a base member 10. Examples of the seeding method include a method using a pipette and an inkjet method.

Next, a first frame member 20 as a frame member for cell culture is superposed on the base member 10, and the respective cells are cultured using a medium appropriate for the cell types, such that a medium M1 is used for the first cells A, a medium M2 is used for the second cells B, a medium M3 is used for the third cells C, and a medium M4 is used for the fourth cells D. Thereby, the cell culture conditions can be coordinated for cells of the same type.

After culturing the cells until the number of cells reaches a predetermined value, the medium is removed, and the first frame member 20 is detached from the base member 10. Thereafter, a second frame member 30 as a frame member for drug evaluation is superposed on the base member 10, solutions having different kinds or concentrations of drugs, such as a first solution N1, a second solution N2, a third solution N3, a fourth solution N4, a fifth solution N5, a sixth solution N6, and a seventh solution N7, are each dispensed into recess clusters, and an assay is performed concurrently. An assay can be performed while causing different kinds of cells to share a solution of the same composition.

In this way, the combination of the recesses α sharing a solution can be freely changed by properly using the first frame member 20 and the second frame member 30 according to the purpose in each step.

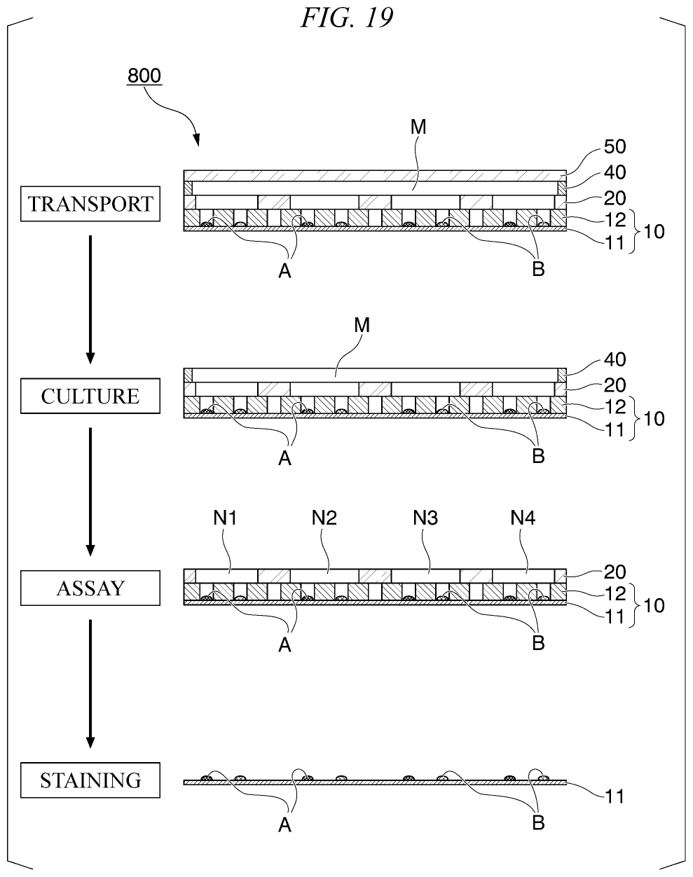

FIG. 19 is a diagram illustrating a method for evaluating a drug as a usage example for a cell culture vessel according to an embodiment of the present invention. In the usage example shown in FIG. 19, transportation, culture, assaying, and staining of cells can be carried out by detaching various members that are laminated in the cell culture vessel in order from the top, according to the purpose in each step.

Specifically, first, first cells A and second cells B are each seeded in a cell culture vessel in which a base member 10, a frame member 20, and an outer frame member 40 are laminated in this order. Next, a medium M is filled up to the top surface of the outer frame member 40 of a liquid reservoir 41, and a lid member 50 is superposed on the outer frame member 40 to produce a cell chip 800. In this state, the cell chip 800 is transported to a target place. By including the lid member 50, the cell chip 800 can be prevented from drying, and the cell chip 800 can be stably transported without spilling the medium in the cell chip 800.

Next, the lid member 50 is detached, and culturing is performed in an environment appropriate for the first cells A and the second cells B. Next, the medium M is removed, the outer frame member 40 is detached, subsequently solutions having different kinds and concentrations of drugs, such as a first solution N1, a second solution N2, a third solution N3, and a fourth solution N4, are each dispensed in recess clusters 5, and an assay is performed concurrently. By detaching the outer frame member 40 and using the frame member 20, an assay can be performed while causing different kinds of cells to share solutions having different compositions.

After completion of the assay, the solution is removed, the frame member 20 and a recess-forming frame member 12 are detached from a substrate 11, and the first cells A and the second cells B are each brought to a state of being disposed on the substrate 11. Thereby, staining of cells and the like can be conveniently carried out, and after staining, the cells can be directly observed and evaluated using a microscope or the like.

### <Cell Chip>

A cell chip according to an embodiment of the present invention includes the above-described cell culture vessel, cells, and a medium. The cells are accommodated in recesses of the cell culture vessel. The medium is filled in the recesses (recess cluster) of the cell culture vessel. The cell chip of the present embodiment can be directly used for an assay such as drug screening.

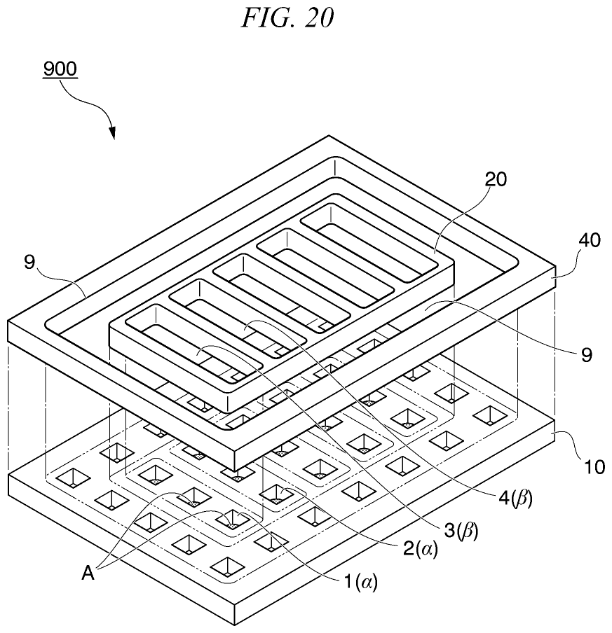

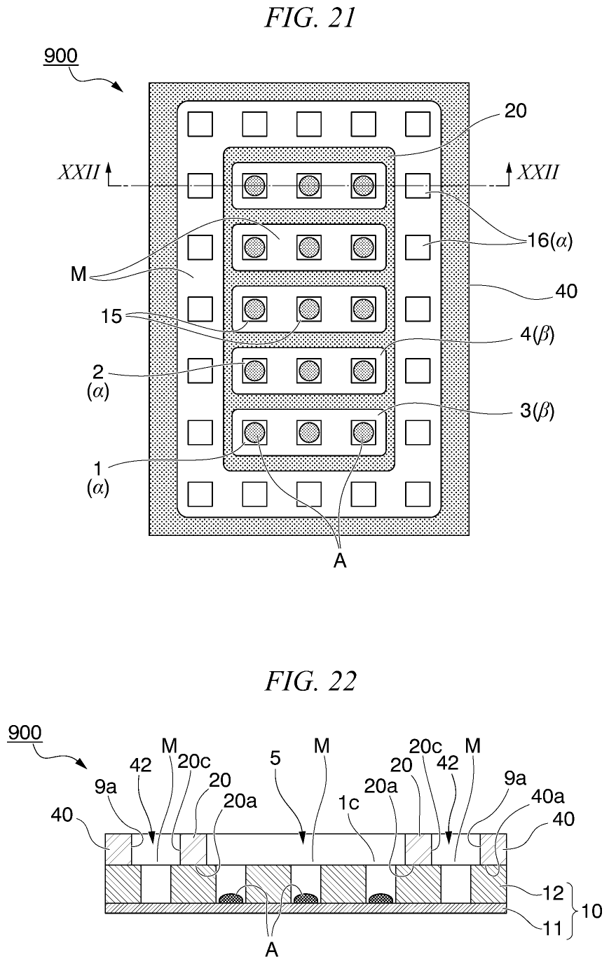

FIGS. 20 to 22 are diagrams illustrating a cell chip 900 according to an embodiment of the present invention. FIG. 20 is an exploded perspective view of the cell chip 900. FIG. 21 is a plan view of the cell chip 900. FIG. 22 is a cross-sectional view of the cell chip 900 cut along a plane that passes through the line XXII-XXII of FIG. 21.

The cell chip 900 shown in FIGS. 20 to 22 includes a cell culture vessel, cells A, and a medium M. The cell A is accommodated in recesses α of the cell culture vessel. The medium M is filled in a recess cluster 5. A frame member 20 and an outer frame member 40 are used in a state of being each stacked on the base member 10.

As shown in FIG. 21, the cell chip 900 has recesses 15 in which cells are accommodated, and recesses 16 in which cells are not accommodated. Furthermore, when a frame member 20 and an outer frame member 40 are stacked on a base member 10, inside a through-hole β of the frame member 20, only the recesses 15 in which cells are accommodated are completely exposed as viewed in plan view. On the other hand, inside a communicating hole 9 included in the outer frame member, only the recesses 16 in which cells are not accommodated are completely exposed around the frame member 20 as viewed in plan view.

Moreover, as shown in FIG. 22, in the cell chip 900, a groove 42 surrounded by the recesses 16 in which cells are not accommodated, an inner wall 9a of the communicating hole 9, and an outer wall 20c of the frame member 20 is formed. Generally, since the medium easily evaporates from the outside of the cell culture vessel, when the groove 42 is formed, the cells A can be prevented from drying.

### (Cells)

Regarding the cells used in the cell chip, the type of the cells and the like are not particularly limited and can be appropriately selected according to the purpose, and taxonomically, the cell chip can be used for all cells, irrespective of, for example, eukaryotic cells, prokaryotic cells, cells of multicellular organisms, and cells of unicellular organisms. These may be used singly, or two or more kinds thereof may be used in combination.

Examples of eukaryotic cells include animal cells, insect cells, plant cells, and fungi.

These may be used singly, or two or more kinds thereof may be used in combination. Among these, animal cells are preferable, and in a case where cells form cell aggregates, adhesive cells having cell adhesiveness to the extent that the cells adhere to one another and are not isolated unless a physicochemical treatment is carried out are more preferable.

The adhesive cells are not particularly limited and can be appropriately selected according to the purpose, and examples thereof include differentiated cells and undifferentiated cells. These may be used singly, or two or more kinds thereof may be used in combination.

Examples of the differentiated cells include hepatocytes, which are parenchymal cells of the liver; stellate cells; Kupffer cells; endothelial cells such as vascular endothelial cells, sinusoidal endothelial cells, and corneal endothelial cells; fibroblasts; osteoblasts; osteoclasts; periodontal ligament-derived cells; epidermal cells such as epidermal keratinocytes; epithelial cells such as tracheal epithelial cells, gastrointestinal epithelial cells, cervical epithelial cells, and corneal epithelial cells; mammary glandular cells; pericytes; muscle cells such as smooth muscle cells and cardiac muscle cells; renal cells; islets of Langerhans cells; nerve cells such as peripheral nerve cells and optic nerve cells; cartilage cells; and bone cells. The adhesive cells may be primary cells collected directly from tissues or organs, or they may be cells that have been passaged several times. These may be used singly, or two or more kinds thereof may be used in combination.

The undifferentiated cells are not particularly limited and can be appropriately selected according to the purpose, and examples include pluripotent stem cells such as embryonic stem cells which are undifferentiated cells, and mesenchymal stein cells having pluripotency; unipotent stem cells such as vascular endothelial precursor cells having unipotency; and induced pluripotent stem cells (iPS cells). These may be used singly, or two or more kinds thereof may be used in combination.

Examples of the prokaryotic cells include eubacteria and archaebacteria.

### (Medium)

The medium used for the cell chip is not particularly limited and may be appropriately selected according to the purpose; however, it is preferable that a medium for cell culture and a buffer solution be included.

A medium is a solution that includes components required for cell culture and maintenance, prevents drying, and creates an external environment such as osmotic pressure, and any medium that is known as a medium can be appropriately selected and used.

The buffer solution is for adjusting the pH according to the cells and the purpose, and any known buffer solution can be appropriately selected and used.

The present invention includes the following aspects.

(1) A cell culture vessel, including a base member having a plurality of recesses; and a frame member having through-holes and configured to be detachable from and attachable to the base member. When the frame member is stacked on the base member, the plurality of recesses and the through-holes communicate with each other, the frame member includes a plurality of kinds of frame members, and the plurality of kinds of frame members have mutually different combinations of the through-holes communicating with the plurality of recesses.

(2) The cell culture vessel according to (1), in which the plurality of recesses include a first recess and a second recess, each of the through-holes communicates with two or more recesses to form a recess cluster surrounded by two or more recesses and the through-hole, and the frame member has a first through-hole that communicates with two or more recesses including the first recess; and a second through-hole that communicates with two or more recesses including the second recess.

(3) The cell culture vessel according to (2), in which a volume of the recess is 10 μL or less, and a volume of the recess cluster is 10 μL or more and 100 μL or less.

(4) The cell culture vessel according to any one of (1) to (3), in which each of the through-holes communicates with three or more recesses.

(5) The cell culture vessel according to any one of (1) to (4), in which inside each of the through-holes, the plurality of recesses and a portion of a top surface of the base member are exposed as viewed in plan view, the through-hole has a first exposed part in which the plurality of recesses are exposed, and a second exposed part that is formed contiguously to the first exposed part and exposes only the top surface of the base member as viewed in plan view, and an area of the top surface of the base member exposed in the second exposed part is larger than a plan view area of the recess.

(6) The cell culture vessel according to (5), in which in the frame member, the plurality of through-holes are arranged along an arrangement axis extending in one direction, and in two through-holes adjoining each other along the arrangement axis, the second exposed part included in one through-hole and the second exposed part included in the other through-hole are disposed, with the arrangement axis interposed therebetween.

(7) The cell culture vessel according to any one of (1) to (6), in which the frame member includes a first frame member and a second frame member, and the first frame member is intended for cell culture, while the second frame member is intended for drug evaluation.

(8) The cell culture vessel according to (7), in which the first frame member is used in a state of being stacked on a top surface of the base member, the second frame member is used in a state of being stacked on a top surface of the first frame member, and when the first frame member and the second frame member are stacked on the base member, two or more of the through-holes included in the first frame member are completely exposed inside the through-holes included in the second frame member, as viewed in plan view.

(9) The cell culture vessel according to any one of (1) to (8), in which the frame member includes an outer frame member having one communicating hole, the communicating hole communicates with the plurality of recesses and the through-holes to form a liquid reservoir surrounded by the plurality of recesses, the through-holes, and the communicating hole, and when the outer frame member is stacked on the base member or the frame member, the plurality of recesses included in the base member and the through-holes included in the frame member are all completely exposed inside the communicating hole included in the outer frame member, as viewed in plan view.

(10) The cell culture vessel according to any one of (1) to (9), further including, on an uppermost surface, a lid member configured to be detachable from and attachable to the frame member.

(11) The cell culture vessel according to any one of (1) to (10), in which the base member has a substrate and a recess-forming frame member that is used in a state of being stacked on the substrate and has a plurality of through-holes for recesses corresponding to the plurality of recesses, and a space surrounded by the substrate and the through-holes for recesses forms the recesses.

(12) The cell culture vessel according to any one of (1) to (11), in which the base member has two or more joining recesses on a top surface thereof, the frame member has two or more joining protrusions corresponding to the joining recesses on a bottom surface thereof, and when the frame member is stacked on the base member, the joining recesses and the joining protrusions are fitted together.

(13) The cell culture vessel according to (12), in which the frame member has two or more recesses for solution trial on a top surface thereof, and in the frame member, the recesses for solution trial and the joining protrusions are disposed at positions where the recesses for solution trial and the joining protrusions overlap each other as viewed in plan view.

(14) A cell chip, including: a cell culture vessel of any one of (1) to (13); cells accommodated in the recesses of the cell culture vessel; and a medium filled in the recesses.

(15) A cell chip, including: a cell culture vessel of (9); cells accommodated in the recesses of the cell culture vessel; and a medium filled in the recesses. The cell chip has recesses in which cells are accommodated, and recesses in which cells are not accommodated, in a periphery of the recesses in which the cells are accommodated. The frame member and the outer frame member are each used in a state of being stacked on the base member, and when the frame member and the outer frame member are stacked on the base member, inside the through-holes included in the frame member, only the recesses in which cells are accommodated are completely exposed as viewed in plan view, and in the periphery of the frame member, which is inside of the communicating hole included in the outer frame member, only the recesses in which cells are not accommodated are completely exposed as viewed in plan view.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the above description, and is only limited by the scope of the appended claims.

### EXPLANATION OF REFERENCES

1 . . . First recess
1a . . . Inner wall
1b . . . Bottom surface
1c . . . Opening
2 . . . Second recess
2a . . . Inner wall
2b . . . Bottom surface
2c . . . Opening
3 . . . First through-hole
3a . . . Inner wall
4 . . . Second through-hole
4a . . . Inner wall
5 . . . Recess cluster
5a . . . inner wall
6 . . . First exposed part
7 . . . Second exposed part
8 . . . Third through-hole
8a . . . Inner wall
9 . . . Communicating hole
9a . . . Inner wall
10 . . . Base member
10a . . . Top surface
11 . . . Substrate
12 . . . Recess-forming frame member
13 . . . First joining recess
13a . . . Bottom surface
14 . . . Second joining recess
14a . . . Bottom surface
15 . . . Recess in which cells are accommodated
16 . . . Recess in which cells are not accommodated
20 . . . (First) frame member
20a . . . Bottom surface (joining side principal surface)
20b . . . Top surface
20c . . . Outer wall
21 . . . First recess for solution trial
21a . . . First joining protrusion
22 . . . Second recess for solution trial
22a . . . Second joining protrusion
30 . . . Second frame member
30a . . . Bottom surface
30b . . . Top surface
40 . . . Outer frame member
40a . . . Bottom surface
40b . . . Top surface
41 . . . Liquid reservoir
42 . . . Groove
50 . . . Lid member
50a . . . Bottom surface
50b . . . Top surface
100,200,300,400,500,600 . . . Cell culture vessel
700,800,900 . . . Cell chip
α . . . Recess
β . . . Through-hole
γ Through-hole for recess
δ . . . Joining recess
ε . . . Joining protrusion
ζ . . . Recess for trial
A . . . (First) cell
B . . . Second cell
C . . . Third cell
D . . . Fourth cell
M . . . Medium
M1 . . . First medium
M2 . . . Second medium
M3 . . . Third medium
M4 . . . Fourth medium
N1 . . . First solution
N2 . . . Second solution
N3 . . . Third solution
N4 . . . Fourth solution
N5 . . . Fifth solution
N6 . . . Sixth solution
N7 . . . Seventh solution
P . . . Pipette
Y . . . Arrangement axis

### PRIOR ART LITERATURE

### Patent Documents

Patent Document 3: Japanese Patent (Granted) Publication No. 4576539

What is claimed is:

1. A cell culture vessel, comprising:
a base member having a plurality of recesses; and
a frame member having through-holes and configured to be detachable from and attachable to the base member,
wherein when the frame member is stacked on the base member, the plurality of recesses and the through-holes communicate with each other,
the frame member includes a plurality of kinds of frame members, and
the plurality of kinds of frame members have mutually different combinations of the through-holes communicating with the plurality of recesses.

2. The cell culture vessel according to claim 1,
wherein the plurality of recesses include a first recess and a second recess,
each of the through-holes communicates with two or more recesses to form a recess cluster surrounded by two or more recesses and the through-hole, and
the frame member has a first through-hole that communicates with two or more recesses including the first recess, and a second through-hole that communicates with two or more recesses including the second recess.

3. The cell culture vessel according to claim 2,
wherein a volume of the recess is 10 μL or less, and a volume of the recess cluster is 10 μL or more and 100 μL or less.

4. The cell culture vessel according to claim 1,
wherein each of the through-holes communicates with three or more recesses.

5. The cell culture vessel according to claim 1,
wherein inside each of the through-holes, the plurality of recesses and a portion of a top surface of the base member are exposed as viewed in plan view,
the through-hole has a first exposed part in which the plurality of recesses are exposed, and a second exposed part that is formed contiguously to the first exposed part and exposes only the top surface of the base member as viewed in plan view, and
an area of the top surface of the base member exposed in the second exposed part is larger than a plan view area of the recess.

6. The cell culture vessel according to claim 5,
wherein in the frame member, the plurality of through-holes are arranged along an arrangement axis extending in one direction, and
in two through-holes adjoining each other along the arrangement axis, the second exposed part included in one through-hole and the second exposed part included in the other through-hole are disposed, with the arrangement axis interposed therebetween.

7. The cell culture vessel according to claim 1,
wherein the frame member includes a first frame member and a second frame member, and
the first frame member is intended for cell culture, while the second frame member is intended for drug evaluation.

8. The cell culture vessel according to claim 7,
wherein the first frame member is used in a state of being stacked on a top surface of the base member,
the second frame member is used in a state of being stacked on a top surface of the first frame member, and
when the first frame member and the second frame member are stacked on the base member, two or more of the through-holes included in the first frame member are completely exposed inside the through-holes included in the second frame member, as viewed in plan view.

9. The cell culture vessel according to claim 1,
wherein the frame member includes an outer frame member having one communicating hole,
the communicating hole communicates with the plurality of recesses and the through-holes to form a liquid reservoir surrounded by the plurality of recesses, the through-holes, and the communicating hole, and
when the outer frame member is stacked on the base member or the frame member, the plurality of recesses included in the base member and the through-holes included in the frame member are all completely exposed inside the communicating hole included in the outer frame member, as viewed in plan view.

10. The cell culture vessel according to claim 1, further comprising, on an uppermost surface, a lid member configured to be detachable from and attachable to the frame member.

11. The cell culture vessel according to claim 1,
wherein the base member has:
a substrate; and
a recess-forming frame member that is used in a state of being stacked on the substrate and has a plurality of through-holes for recesses corresponding to the plurality of recesses, and
a space surrounded by the substrate and the through-holes for recesses forms the recesses.

12. The cell culture vessel according to claim 1,
wherein the base member has two or more joining recesses on a top surface thereof,
the frame member has two or more joining protrusions corresponding to the joining recesses on a bottom surface thereof, and
when the frame member is stacked on the base member, the joining recesses and the joining protrusions are fitted together.

13. The cell culture vessel according to claim 12,
wherein the frame member has two or more recesses for solution trial on a top surface thereof, and
in the frame member, the recesses for solution trial and the joining protrusions are disposed at positions where the recesses for solution trial and the joining protrusions overlap each other as viewed in plan view.

14. A cell chip, comprising:
a cell culture vessel of claim 1;
cells accommodated in the recesses of the cell culture vessel; and
a medium filled in the recesses.

15. A cell chip, comprising:
a cell culture vessel of claim 9;
cells accommodated in the recesses of the cell culture vessel; and
a medium filled in the recesses,
wherein the cell chip has
recesses in which cells are accommodated, and recesses in which cells are not accommodated, in a periphery of the recesses in which cells are accommodated,
the frame member and the outer frame member are each used in a state of being stacked on the base member, and
when the frame member and the outer frame member are stacked on the base member, inside the through-holes included in the frame member, only the recesses in which cells are accommodated are completely exposed as viewed in plan view, and in the periphery of the frame member, which is inside of the communicating hole included in the outer frame member, only the recesses in which cells are not accommodated are completely exposed as viewed in plan view.
